(19) United States
(12) Patent Application Publication
(10) Pub. No.: US 2005/0147964 A1
(43) Pub. Date: Jul. 7, 2005

(54) Methods for identifying a peptide that binds a geometrical shape

(75) Inventors: Mineo Yamakawa, Campbell, CA (US); Joe Kosmoski, Los Altos, CA (US); C. Deane Little, Los Altos, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Intel Corporation, Santa Clara, CA (US)

(21) Appl. No.: 10/749,532

(22) Filed: Dec. 30, 2003

(51) Int. Cl. C12Q 1/70; G01N 33/53; C12P 19/34

(52) U.S. Cl. 435/5; 435/7.1; 435/91.2

(57) ABSTRACT

Provided herein are methods, such as phage display assays, for bioengineering peptides that bind to geometrically and/or atomically structured molecular surfaces such as, for example, flat surfaces, smooth curved surfaces, as well as surfaces with periodic, random, or fractal atomic configurations. Surface-binding peptides are provided that are identified using the phage-display methods. Furthermore, scanning probe microscopy (SPM) substrates, biosensors, biochips, and electrodes are provided that include the surface-binding peptides.

### BACKGROUND OF THE INVENTION

1. Field of the Invention

The invention relates generally to selective recognition and more specifically to selective recognition of geometrical shapes.

2. Background Information

Advances in medicine such as new diagnostic techniques require highly sophisticated bioreactors, microelectronics, microelectrodes, and biomolecular analysis techniques, for example for use in sensitive biosensors. Powerful analytical techniques such as scanning probe microscopy (SPM) have been developed, along with powerful tagging techniques in which very small structures called nanotags, are used to identify larger molecules such as biomolecules. The detection of nanotags and biomolecules using these powerful analytical techniques requires binding of the nanotags and biomolecules to substrates that are anatomically flat and sometimes highly hydrophobic, which are difficult surfaces for nanotag and biomolecular binding. Thus, a need exists for methods and compositions that can be used to facilitate binding of nanotags and biomolecules to anatomically flat and hydrophobic substrates.

In general, attachment and binding of biomolecules such as peptides and polypeptides to specific materials and substrates involve, for example, chemical adsorption and hydrophobic/hydrophilic interactions, or chemical reactions such as binding of a thiol group of a peptide to gold. However, often peptide or polypeptide binding to a very hydrophobic and atomically flat surface is very difficult often resulting in denaturization or conformational changes of the peptide structure. Presently no good solutions for reliable binding of peptides or polypeptides to such surfaces are known. For example highly ordered/oriented pyrolytic graphite (HOPG) is a popular and common substrate used for holding deposits of samples for SPM scanning. However, peptides and polypeptides tend to be non-uniformly deposited after being dried without any specific protocols. Thus, a need exists for peptides that specifically bind to atomically flat surfaces, and for methods to identify these peptides.

### BRIEF DESCRIPTION OF THE DRAWINGS

FIG. 1 diagrammatically illustrates a combinatorial display of peptides, wherein a specific peptide binds a flat surface.

### DETAILED DESCRIPTION OF THE INVENTION

Methods for bioengineering (e.g., discovering/identifying, designing, and/or synthesizing) molecules that can bind to geometrically and/or atomically structured (e.g., flat, fractal or random) molecular surfaces of a structure/substrate, are provided herein. This type of binding is required in a variety of analytical preparations including SPM (e.g., AFM and STM) scanning of samples such as nanocodes, which can be, for example, peptide based molecules such as enzymes, glycoproteins, oligopeptides, or synthetic nanotags. Also, immobilizing peptide-based biomolecules such as antibodies (i.e., glycoproteins) and reaction enzymes (e.g., kinases and phosphorylases) for biosensors, bioreactors, microelectronics, and microelectrode requires this type of binding.

Phage display technology allows the rapid discovery, identification, and selection of target peptides with appropriate chemical and/or physical characteristics, compared to a selection by theoretical or intuitive guesswork, which takes a significant amount of time and effort, if successful at all. Thus, methods provided herein utilize phage display to discover new materials for use in analytical preparations and devices, and new circuit structures for processing and decoding information. Furthermore, compositions and methods provided herein include molecular combinations of two or more defined activities to provide a family of building blocks to create nano-molecular scaffoldings and attachment sites. These nano-molecular scaffolding and attachment sites allow reading, decoding, and computations based on (bio)molecules and allow creation of new electronic circuits, for example.

Accordingly, methods to bio-engineer peptide-based molecules that possess specific chemical and/or physical affinities for geometrically or atomically specific structured/patterned surfaces, is provided. The peptides are useful, for example, for attaching nanocodes to a substrate surface for enabling reliable and accurate barcode reading (i.e., encoding and decoding information in nanotags).

Accordingly, in one embodiment a method is provided for identifying a peptide that binds to a surface having a target geometrical shape or a target atomic configuration, that includes contacting the surface having the target geometrical shape with a library of peptides or polypeptides, and identifying the peptides or polypeptides that bind to the surface having the target geometrical shape or atomic configuration. In certain aspects, each peptide or polypeptide is associated with an encoding polynucleotide. These aspects facilitate isolation and sequencing of the encoding polynucleotide.

In another embodiment, a method is provided for identifying a peptide that binds to a surface having a target geometrical shape, that include contacting the surface having the target geometrical shape with a phage display library under reaction conditions, wherein the phage express a peptide, and identifying peptides that bind to the surface having the target geometrical shape.

In certain aspects, the library of peptides or polypeptides is generated using straight chemical synthesis instead of using a phage display library (See e.g. on the world wide web at dkfz-heidelberg.de/cbpl/; and Houghten et al., Nature, 354:84 (1991)). For example, a chip-based peptide library can be screened for peptides that bind a surface having a target geometrical shape. Alternatively, bacteria can be used for creating combinatorial peptides, as is known in the art. The library, for example, can include more than 1,000, 10,000, 100,000, 1,000,000, or 10,000,000 unique peptides. In one specific, non-limiting example, the library includes 34 million hexa-peptides.

In another aspect, a polypeptide that binds to a surface having a target geometrical shape is identified by immunizing a mammalian organism, such as, for example a rodent or a human, with a surface having the target geometrical shape, and identifying antibodies against the target geometrical shape that are produced by the organism. Methods for immunizing mammalian organisms and identifying antibodies are known in the art. Also, fragments of the antibody, such as Fab fragments, can be isolated. Furthermore, antigen binding regions of identified antibodies that bind the target geometrical shape can be isolated.

A geometrical shape is a characteristic surface configuration. An atomic configuration is the arrangement of atoms of a surface, and optionally the surrounding solvation sphere. In certain aspects, the target geometrical shape of the surface is a flat surface, or a very flat surface. In other aspects, the target geometrical shape of the surface is a smooth surface, such as a smooth, curved surface. For example, the smooth, curved surface can be that of a nanotube. In other examples, the anatomic configuration is periodic, fractal, or random. Furthermore, the surface, in certain examples, is hydrophobic.

A flat surface is an even surface that is free from roughness, irregularities, or projections. A flat surface can be non-curved or curved. A smooth surface is a surface that is free from roughness, irregularities, or projections. A smooth, curved surface is a surface that deviates from straightness in a continuous way that is free from roughness, irregularities, or projections. A structure with a periodic atomic configuration is a structure that is composed of a specific molecular configuration that occurs at repeated intervals. A surface with a fractal atomic configuration is a surface that is composed of, an unusual number of dimensions (e.g., 2.381 dimensions) and that looks essentially the same, regardless of the magnification. A surface with a random atomic configuration is a surface that is composed of molecules that have no specific pattern or organization.

As is known, any surface with properties/structures similar to, for example, annealed gold, HOPG, and/or Teflon® are considered to be atomically very flat by SPM microscopy. A silicon surface is considered to be “flat” by SPM microscopy. Fractal dimensions are an index of complexity and/or “flatness” or “smoothness” depending on the scales if fractal dimension is constant.

A surface that is bound by surface-binding peptides and polypeptides disclosed herein, can be, for example, a flat surface or another smooth surface, such as a smooth, curved surface. Furthermore, the surface can include organic and/or inorganic components. For example, the surface can be a substrate for an analytical or measurement device such as a substrate for scanning probe microscopy (SPM), or any other analytical or measurement device that can utilize a flat surface or a smooth, curved surface. An SPM substrate can be a graphite substrate, for example, such as a highly ordered pyrolytic graphite (HOPG) substrate. In another aspect, the surface is a carbide or graphite electrode, which can be used for example, in nanotube production. In other examples, the surface-binding peptides identified herein bind semiconductor surfaces.

The surface can be composed of a wide-variety of components. For example, the surface can be composed, at least in part, of boron nitride, lead sulfide, zinc selenide, cadmium selenide, cadmium sulfide, gallium arsenide, aluminum arsenide, zinc sulfide, gallium nitrate, indium phosphate, or gallium arsenide. In other aspects, the surface includes mica, silicon, or annealed gold. In other examples, the surface is composed, at least in part, of Teflon®.

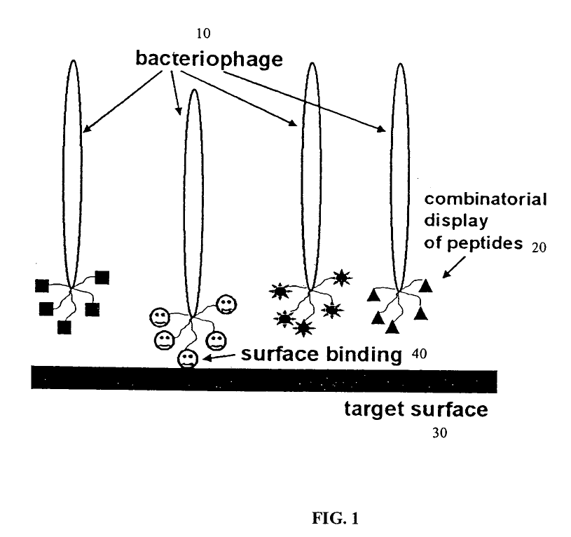

Phage display library generation and screening in general are known in the art (See e.g., Barbas, C., et al., “Phage Display A Laboratory Manual,” Cold Spring Harbor (2001); and Kay et al., Methods 24, 240-246 (2001)). Phage display libraries can be constructed using known methods, or they can be purchased (e.g., from New England BioLabs (Beverly, Mass.)). As shown in FIG. 1, the methods provided herein involve a binding assay that includes contacting a library of phage 10 (e.g., M13 filamentous phage), each displaying a different exogenous peptide sequence 20 on the surface of the bacteriophage 10, to a target surface 30, such as a graphite surface, with a specific geometrical pattern or geometrical shape such as an atomically flat structure 30. After exposure of the phage 10 to the target surface 30 for an appropriate incubation period to allow binding, unbound phage 10 are washed away and the specifically bound phage 10 are eluted or specifically removed.

Eluted phage are amplified, and the process is repeated, for example for a total of 2-20 rounds, more specifically, for example for 2-10 rounds, and even more specifically, for example for 3-4 rounds. The screening of phage display libraries for surface-binding peptides through multiple rounds of screening, as disclosed herein, is referred to as biopanning. Phage are amplified using known methods, for example by reinfecting host bacteria with the phage and culturing the reinfected host bacteria. In certain embodiments, DNA of identified phage is amplified by using an in vitro amplification procedure such as the polymerase chain reaction PCR.

The incubation period for binding of the phage to the surface can be any typical incubation period for a phage display assay, such as, for example, about 5 minutes to about 1 day, or more specifically from 15 minutes to 4 hours. In a specific example the incubation occurs for 2 hours. Elution or removal of specifically bound phage typically involves use of harsh conditions to inhibit the interaction of the peptide to the substrate. For example, an elution solution of an extreme pH (e.g., 1-3, 8-12), or which includes a denaturing agent such as urea, and/or a protease, such as trypsin, can be used.

Phage display engineering has been used to discover peptides that bind to nanoparticles/quantum dots (S. Lee, et al., Science 2002, 296:892.). Numerous groups have reported that “plate binders” are in vast excess to other desired activities, yet these plate binders have been overlooked as desirable lead compounds. (Barbas, C., et al., “Phage Display A Laboratory Manual,” Cold Spring Harbor, 2001). Furthermore, as illustrated in the examples herein, peptides have been identified using phage display methods that bind to plastics surfaces.

Peptides or polypeptides that bind to a flat surface or a smooth, curved surface are referred to herein as “surface-binding peptides or polypeptides” or “binder peptides or polypeptides.”

Biomolecules as used herein include, but are not limited to, nucleic acids, peptides, proteins, polysaccharides, and combinations thereof, as well as other biological substrates, inhibitors, activators, ligands, hormones, or cytokines.

“Nucleic acid” encompasses DNA, RNA (ribonucleic acid), single-stranded, double-stranded or triple stranded and any chemical modifications thereof. Virtually any modification of the nucleic acid is contemplated. A “nucleic acid” can be of almost any length, from oligonucleotides of 2 or more bases up to a full-length chromosomal DNA molecule. Nucleic acids include, but are not limited to, oligonucleotides and polynucleotides. A “polynucleotide” as used herein, is a nucleic acid that includes at least 25 nucleotides.

As used herein, the term “specific binding pair member” refers to a molecule that specifically binds or selectively hybridizes to another member of a specific binding pair. Specific binding pair member include, for example, an oligonucleotide and a nucleic acid to which the oligonucleotide selectively hybridizes, or a protein and an antibody that binds to the protein. A “target” or “analyte” molecule includes, but is not limited to, a nucleic acid, a protein, a lipid, and a polysaccharide.

In certain aspects, each round of evolution (or biopanning) of methods disclosed herein, is designed to fit the parameters of “the common denominator principle”. In brief, the principle states that desired elements are present in every evolution step, although presented differently, sometimes by eliminating undesired elements.

Accordingly, in certain aspects, methods disclosed herein utilize combinatorial directed evolution. According to combinatorial directed evolution methods, peptides are identified using several rounds of biopanning. In certain aspects, at least 2, 3, 4, 5, 6, 7, 8, 9, 10, 15, 20, and 25 rounds of biopanning are performed by repeating the contacting, identifying, and amplifying steps. During each successive round of biopanning, for example, the reaction conditions can be made more stringent than the prior round. More stringent conditions are conditions in which a higher affinity (i.e. a lower dissociation constant) of the peptide for the surface is required for the peptide to bind to the surface. Parameters such as pH, ionic strength, concentration of metal ion, and temperature changes, for example, can affect binding of a peptide to a surface. More stringent conditions can be provided, for example, by acidifying or alkylating an incubation buffer for the binding reaction or by increasing the temperature of the incubation.

If a specific peptide sequence that has negative affinities to other specific geometrical structures of a material surface is required, the binding assay can include both positive and negative selection pressure. Positive selection is generally used to select for affinity whereas negative selection often enhances specificity. Peptide sequences with non-specific binding can also be eliminated.

As an example of an aspect that uses negative selection to remove unwanted peptides, peptides identified in one or more rounds of screening of a surface with a target geometrical shape or atomic configuration, are placed in contact with a surface with an identical or substantially identical chemical composition but an undesirable geometrical shape and/or atomic configuration. Substantially identical chemical compositions include the same major chemical constituents but can include minor differences in chemical constituents. Phage that do not bind the surface with the undesirable geometrical shape and/or atomic configuration, are collected and optionally amplified and subjected to additional rounds of biopanning. For example, the surface with the undesirable geometrical shape or atomic configuration, can be composed of crystals and therefore have a surface that is not flat. Phage that bind to the crystals can be eliminated. The rounds of selection and removal can be repeated to increase the binding strength and/or specificity of identified phage. Combinations of desirable/undesirable characteristics for screening phage libraries include, for example, screening for peptides that bind a flat geometry or a smooth, curved geometry, but do not bind a crystal, or screening for peptides that bind a periodic atomic configuration but not a random atomic configuration.

In certain aspects, in addition to the desired geometrical shape and/or atomic configuration, the target surface can have additional desired properties. For example, the surface can have certain chemical properties in addition to a desired geometrical shape and/or atomic configuration. In these aspects, for example, phage can be identified that bind to a flat surface or a smooth, curved surface but only if it is hydrophobic, negatively charged, or positively charged.

As a specific example, phage can be screened for binding to flat, hydrophobic surfaces in general. Historically, it has been difficult to identify peptides that bind to a surface with these characteristics. Accordingly, in this specific aspect, different flat, hydrophobic surfaces can be used to identify a peptide that binds many different flat and hydrophobic surfaces. A peptide with these characteristics is valuable for example, to assist in coating of biomolecules and/or nanocodes to SPM substrates.

In certain aspects, the phage is amplified using a sloppy amplification reaction during one or more rounds of biopanning. A “sloppy amplification reaction” is a reaction utilizing a process known as sloppy PCR, error-prone PCR or mutagenic PCR (PCR Primer, A Laboratory Manual, Cold Spring Harbor Laboratory Press (1995)). Error-prone, sloppy, or mutagenic PCR is a process for performing the polymerase chain reaction under conditions where the copying fidelity of the DNA polymerase is low, such that a high rate of point mutations is obtained along the entire length of the PCR product. This process increases the diversity of peptides in the phage library after initial round(s) of biopanning identify at least weakly binding peptides.

Accordingly, methods of the present invention involve the use of phage display technology to identify, through combinatorial directed evolution, specific amino acid sequence(s) of a peptide that preferentially bind to a specific material surface of a geometrically distinct structure, such as a substrate for a measurement devices or analytical instrument that utilize substrates of particular shapes or atomic configuration, such as scanning probe microscopy (SPM). In certain aspects of the invention, the surface to which the surface-binding peptides or polypeptides of the present invention bind, is composed, at least in part, of an elemental carbon-containing molecule. The term “elemental carbon-containing molecule” generally refers to allotropic forms of carbon. Examples include, but are not limited to, diamond, graphite, activated carbon, carbon₆₀, carbon black, industrial carbon, charcoal, coke, and steel. Other examples include, but are not limited to carbon planchet, highly ordered pyrolytic graphite (HOPG), single-walled nanotube (SWNT), single-walled nanotube paste, multi-walled nanotube, multi-walled nanotube paste as well as metal impregnated carbon-containing materials.

The surface to which the surface-binding peptides and polypeptides disclosed herein bind can be a substrate or a surface of a substrate. A “substrate” can be a microfabricated solid surface to which molecules attach through either covalent or non-covalent bonds and includes, e.g., silicon, Langrnuir-Bodgett films, functionalized glass, germanium, ceramic, silicon, a semiconductor material, PTFE, carbon, polycarbonate, mica, mylar, plastic, quartz, polystyrene, gallium arsenide, gold, silver, metal, metal alloy, fabric, and combinations thereof capable of having functional groups such as amino, carboxyl, thiol or hydroxyl incorporated on its surface. Similarly, the substrate may be an organic material such as a protein, mammalian cell, antibody, organ, or tissue with a surface to which a biologic material may attach. The surface may be large or small and not necessarily uniform but should act as a contacting surface (not necessarily in monolayer). The substrate includes a contacting surface that may be the substrate itself or a second layer (e.g., substrate or biologic material with a contacting surface) made of organic or inorganic molecules and to which organic or inorganic molecules may contact. Regardless of its specific composition, a surface according to the present invention is a target geometrical shape or atomic configuration, as disclosed herein.

Previous development of self-assembly monolayers utilize a bi-phasic kinetic model in which a surfactant is absorbed to a surface due to both affinity for the surface and already bound surfactants (Ulman, A., “Formation and Structure of Self-Assembly Monolayers,” Chem. Rev. 1996, 96:1533-1554). The methods disclosed herein provide the advantage that the interdependence of the two binding events, one to the surface and the other to already bound surfactants, can be controlled though selective pressure for either. Accordingly, in certain aspects, the surface used for a method for identifying a peptide disclosed herein is composed at least in part, or is bound by, a surfactant. An identified phage expressing a surface-binding peptide can bind to the surfactant or to both the surface and the surfactant. Alternatively, the surfactant, for example, can be a monolayer that covers the surface such that peptides expressed on the phage bind to the surfactant but cannot bind to the surface.

As discussed above, methods disclosed herein include selection and identification of high affinity binding sequences through appropriate assays of the peptide, also referred to as peptide ligands, with affinities to specific geometrical structures of a material surface by biopanning (i.e., an in vitro selection process). In certain methods disclosed herein, a physical linkage exists between peptide from a large library of random peptide sequences and a nucleic acid encoding each sequence. Therefore, After biopanning, individual clones are isolated and sequenced using methods well known in the art. According to these methods, a particular DNA sequence encoding a peptide that demonstrates specific binding to a specific geometrical shape or atomic configuration-binding is identified.

The resulting selected DNA sequences are used to identify corresponding amino acid sequences of a surface-binding peptide. The identified peptide using methods disclosed herein, can be separately synthesized (e.g., by peptide synthesizer) and its positive and/or negative binding affinities and binding specificity can be confirmed by appropriate assays as will be understood.

Accordingly, in another embodiment an isolated peptide or polypeptide is provided that binds a flat surface, a smooth surface (e.g., a smooth, curved surface) or a surface with a desired atomic configuration. The identified peptide can include natural or non-natural amino acids (i.e. unnatural amino acids), or combinations thereof. Numerous non-natural amino acids and methods for incorporating such non-natural amino acids into peptide chains are known in the art and can be used with the methods herein (See e.g., Hohsaka et al., Nucleic Acids Research, 29, 17 3646-3651 (2001)). Non-natural amino acids can include almost any group as the R group. In certain aspects, the non-natural amino acid is an isotopic analog, such as C13. C13 amino acids can be used, for example, for NMR experiments. In certain aspects, non-natural amino acids are incorporated into a peptide by using specifically-designed and charged tRNAs that recognize stop codons. In more specific non-limiting examples, the peptide can include Nitrophenylalanine (nitroPhe), -Nitrobenzoxadiazolyl-L-lysine residues, or the selenium-containing tryptophan “analog” b-selenolo[3,2-b]pyrrolylalanine, or any other non-natural amino acid known in the art. Furthermore, in another embodiment, an isolated phage is provided, that includes the surface-binding peptide or polypeptide.

In certain aspects, the surface-binding peptides and/or polypeptides are bound to nanoparticles such as carbon nanotubes. The surface-binding peptides and/or polypeptides bound to nanotubes can be used, for example, to bind an array of the nanotubes to a substrate. The nanotube arrays bound to a substrate can be used in a variety of applications, including, but no limited to, fabrication of miniature electronic, chemical and molecular devices, probes for use in scanning probe microscopy, molecular wires, incorporation into ultrafast random access memory (Rueckes et al., Science 289:94, 2000), field-effect transistors, single electron transistors, field emitter arrays, flat screen panels, electromechanical transducers, molecular switches, and any other known use for carbon nanotube arrays.

The methods and compositions disclosed herein can be used to provide a “mix and match” combinatorial application of desired activities to create a “toolbox” or kit of assembly units. Thus, after identifying a particular surface-binding peptide sequence by phage display, the identified peptide can be replicated into multiple linked units of the same (or similar) or different identified surface-binding peptide sequences. Recombinant DNA technologies can be used to construct coding sequences that encode these peptides, or the peptides can be directly synthesized, as will be understood. For example, if a certain peptide sequence, A-B-C-D-E (where A-E denote amino acids) is discovered to be a good surface binder, a variety of linked permutations of the peptide, such as (A-B-C-D-E)-(A-B-C-D-E)-(A-B-C-D-E)-(A-B-C-D-E), can be used. Various linkers can be used to attach such peptides to create “extended” surface binder peptides. Many linkers are known in the art and can be used. Therefore, the identified surface-binding peptides disclosed herein can be used as stand-alone specific surface-binding peptide sequences as well as “structures” containing multiple (e.g., two or more, three or more, four or more, five or more) chemically linked sequences that can include the same or similar sequence.

Accordingly, in another embodiment, an isolated peptide or polypeptide that includes at least two peptide units, is provided, wherein each peptide unit specifically binds a target geometrical shape or atomic configuration. In certain aspects, the isolated peptide or polypeptide is a recombinant peptide or polypeptide in that it includes about 2, 3, 4, 5, 6, 7, 8, 9, 10, 11, 12, 13, 14, 15, 20, 25, 50, 100 peptide units that are not included in a tandem configuration in a known, natural protein. In certain examples, at least 2 of the peptide units include a different amino acid sequence. As used herein, “about” means within ten percent of a value. For example, “about 100” would mean a value between 90 and 110. The isolated peptide or polypeptide provided herein is typically identified using the method for identifying a peptide disclosed above, and individual surface-binding peptides can include, for example, between about 2 and 100 amino acids, between about 5 and 50, and more specifically, for example, between about 7 and 20 amino acids.

In another aspect, the isolated peptide or polypeptide can be associated with a nanocode. In fact, surface-binding peptides or polypeptides can become a supportive part of an encoded nanocode, or other type of nanotag. In another aspect, the peptide units are linked by a linkage other than a peptide bond, for which many are known in the art, including, for example, Isocynate, phosphoramidite, carboxide, glycol, and azide. As disclosed above, the peptide can be associated with a nanocode.

Surface-binding peptides identified using methods disclosed herein, can be used for a variety of applications. As indicated above, the surface-binding peptides can be used in substrates for scanning probe microscopy (SPM), in biosensors, in electrodes and in semiconductors, for example. Accordingly, in another embodiment, a scanning probe microscopy (SPM) substrate is provided that has a flat surface or a smooth, curved surface, to which a surface-binding peptide or polypeptide is bound. In certain aspects, surface-binding peptides are bound to the substrate in an ordered arrangement.

Any of the many known SPM substrates can be used with the present invention, as long as the substrate has a flat surface or a smooth, curved surface, or a surface with a periodic, fractal, or random atomic configuration. Therefore, the surface, for example, can be composed of glass, ceramic, plastic, polystyrene, polypropylene, polyethylene, polycarbonate, PTFE (polytetrafluoroethylene), PVP (polyvinylpyrrolidone), germanium, silicon, quartz, gallium arsenide, gold, silver, nylon, nitrocellulose or any other material known in the art that is capable of acting as an SPM substrate and having a flat surface or a smooth, curved surface, or a surface with a periodic, fractal, or random atomic configuration. In certain embodiments of the invention the surface is a glass slide or cover slip. In another specific non-limiting example, the surface is a carbon lattice in a fullerene building block structure such as corannulene.

In certain aspects, the SPM substrate is a graphite substrate. In another aspect, the surface-binding peptide or polypeptide bound to the SPM substrate is associated with a biomolecule such as a polypeptide, a polynucleotide, a carbohydrate, or a combination thereof. For example, the surface-binding peptide or polypeptide bound to the SPM substrate can be associated with an enzyme or an antibody. The surface-binding peptide or polypeptide is typically identified using the methods disclosed herein before being bound to the SPM substrate. Furthermore, as disclosed herein, the peptide can include between about 2 and 100, about 5 to 50, and more specifically between about 7 and 20 amino acids, for example.

In another aspect, a biomolecule that is associated with a surface-binding peptide that is bound to an SPM substrate, is bound to a nanoparticle also referred to as a nanotag, such as a nanocode. A “nanocode” is a composition that can be used to detect and/or identify a probe physically associated with the nanocode. In non-limiting examples, a nanocode includes one or more submicrometer metallic barcodes, carbon nanotubes, fullerenes or any other nanoscale moiety that can be detected and identified by scanning probe microscopy. Nanocodes are not limited to single moieties, and in certain embodiments of the invention a nanocode can include, for example, two or more fullerenes attached to each other. Where the moieties are fullerenes, they can, for example, consist of a series of large and small fullerenes attached together in a specific order. The order of differently sized fullerenes in a nanocode can be detected by scanning probe microscopy and used, for example, to identify an attached probe. Nanocodes can be used in many different methods, for example methods such as, but not limited to, polynucleotide sequencing, immunoassays, single nucleotide polymorphism (SNP) detection, specific genotype detection, and ligand binding, as well as personal ID and security protocols.

Accordingly, in another embodiment a method is provided, wherein a surface-binding peptide or polypeptide is contacted with an SPM substrate having a flat surface, wherein the surface-binding peptide binds to the flat surface. Furthermore, the bound surface-binding peptide or polypeptide can be functionalized and contacted with a nanoparticle or a biomolecule or a combination thereof, wherein the nanoparticle or biomolecule or combination thereof, bind to the surface-binding peptide or polypeptide. A population of surface-binding peptides or polypeptides can be aligned on the SPM substrate to align biomolecules that bind to the surface-binding peptides or polypeptides.

Methods for functionalizing peptides are known in the art. For example a bi-functional linker can be added using a bifunctional cross-linking reagents (e.g., available from Sigma-Aldrich, St. Louis, Mo.). The bifunctional cross-linking reagents can be divided according to the specificity of their functional groups, e.g., amino, guanidino, indole, or carboxyl specific groups. Of these, reagents directed to free amino groups are popular because of their commercial availability, ease of synthesis and the mild reaction conditions under which they can be applied. Exemplary methods for cross-linking molecules are disclosed in U.S. Pat. Nos. 5,603,872 and 5,401,511. Cross-linking reagents include glutaraldehyde (GAD), bifunctional oxirane (OXR), ethylene glycol diglycidyl ether (EGDE), and carbodiimides, such as 1-ethyl-3-(3-dimethylaminopropyl) carbodiimide (EDC).

If necessary, an appropriate immobilization and dispersion technique can be used to improve the SPM analysis. For example, in SPM methods a substrate surface treatment such as thiol-gold, polylysine, silanization/AP-mica, as well as Mg²⁺ and/or Ni²⁺ (See e.g., Proc. Natl. Acad. Sci. USA 94:496-501 (1997); Biochemistry 36:461 (1997); Analytical Sci. 17:583 (2001); Biophysical Journal 77:568 (1999); and Chem. Rev. 96:1533 (1996)) can be used to uniformly disperse and immobilize a surface-binding peptide or polypeptide before binding a biomolecule to the peptide or polypeptide. Furthermore, peptide sequences can be identified using methods disclosed herein, that give rise to single or multiple dimension matrices along the surface. Not to be limited by theory, these structures are more likely to form considering biphasic surface chemistry kinetics observed with thiolated probes on gold surfaces.

Scanning probe microscopy (SPM) is well known in the art. Examples of SPM include scanning tunneling microscopy (STM), atomic force microscopy (AFM), lateral force microscopy (LFM), and chemical force microscopy (CFM). Other SPM modes that could benefit from the surface-binding peptides and polypeptides disclosed herein include magnetic force microscopy (MFM), high frequency MFM, magnetoresistive sensitivity mapping (MSM), electric force microscopy (EFM), scanning capacitance microscopy (SCM), scanning spreading resistance microscopy (SSRM), tunneling AFM and conductive AFM.

In another embodiment, a biosensor is provided that includes a substrate having a flat surface, or a smooth, curved surface, wherein a surface-binding peptide or polypeptide is bound to the flat surface or smooth, curved surface of the biosensor. In one aspect, the surface-binding peptide or polypeptide is associated with a biomolecule such as a polypeptide, a polynucleotide, a carbohydrate, or a combination thereof. For example, the peptide or polypeptide can be associated with an enzyme or an antibody. The surface-binding peptide or polypeptide is typically identified using the methods disclosed herein before it is associated with the biosensor. Furthermore, as disclosed herein, the peptide can include between about 2 and 100, about 5 and 50, or more specifically, for example between about 7 and 20 amino acids.

Accordingly, in another embodiment a method is provided, wherein a surface-binding peptide or polypeptide is contacted with a biosensor surface having a flat surface wherein the surface-binding peptide binds to the flat surface. Furthermore, the bound surface-binding peptide or polypeptide can be functionalized and contacted with a specific binding pair member, wherein the specific binding pair member binds to the surface-binding peptide or polypeptide. For example, surfaces with a bound surface-binding peptide or polypeptide that is contacted with a specific binding pair member, can be used to form a substrate for an ELISA assay or can be formed based on a modified ELISA assay.

In general, biosensors consist of two components: a highly specific recognition element and a transducing structure that converts the molecular recognition event into a quantifiable signal. Signal transductions are generally accomplished with electrochemical, field-effect transistor, optical absorption, fluorescence or interferometric devices. Biosensors have been developed to detect a variety of biomolecular complexes including oligonucleotide pairs, antibody-antigen, hormone-receptor, enzyme-substrate and lectin-glycoprotein interactions. Flat or smooth, curved surfaces of biosensors, or surfaces of other target geometries, can be coated with surface-binding peptides or polypeptides in order to facilitate attachment of biomolecular specific binding pair members. The surface-binding peptides and polypeptides can be bound to surfaces of either the recognition element or the transducing element.

In another embodiment, A biochip is provided that includes a substrate having a flat surface, or smooth, curved surface, wherein a surface-binding peptide or polypeptide is bound to the flat surface or smooth, curved surface of the biochip. In one aspect, the surface-binding peptide or polypeptide is associated with a biomolecule such as a polypeptide, a polynucleotide, a carbohydrate, or a combination thereof. For example, the peptide or polypeptide can be associated with an enzyme or an antibody. The surface-binding peptide or polypeptide is typically identified using the methods disclosed herein before it is associated with the biosensor. Furthermore, as disclosed herein, the surface-binding peptide or polypeptide can include between about 2 and 100, about 5 and 50, or more specifically, for example between about 7 and 20 amino acids.

Accordingly, in another embodiment a method is provided, wherein a surface-binding peptide or polypeptide is contacted with a biochip surface having a flat surface, or smooth, curved surface, wherein the surface-binding peptide binds to the flat surface, or smooth, curved surface. Furthermore, the bound surface-binding peptide or polypeptide can be functionalized and contacted with a specific binding pair member, wherein the specific binding pair member binds to the surface-binding peptide or polypeptide. As such, the methods provide for a biochip with a surface-binding probe attached to the biochip surface and optionally also bound to a specific binding pair member. The biochips can be used, for example, to identify an analyte that binds the specific binding pair member.

Non-limiting examples of substrates that can be used with biochip embodiments include glass, silica, silicate, PDMS (poly dimethyl siloxane), silver or other metal coated substrates, nitrocellulose, nylon, activated quartz, activated glass, polyvinylidene difluoride (PVDF), polystyrene, polyacrylamide, other polymers such as poly(vinyl chloride) or poly(methyl methacrylate). Non-limiting examples of assays that can be performed on biochips that include the surface-binding peptides of the present invention include footprinting assays using Fenton chemistry, which is amenable to solid phase activity, and ELISA assays. For each of these examples, catalytic activities can be associated with the biochip through the surface-binding peptides.

In another embodiment, a graphite or carbide electrode is provided, that has a flat surface or a smooth, curved surface, wherein a surface-binding peptide or polypeptide is bound to the flat surface or the smooth, curved surface of the graphite or carbide electrode. Graphite and carbide electrodes are well known in the art as being useful, for example, in the synthesis of carbon nanotubes, such as by Krätschmer are methods (W. Krätschmer, W. et al., Nature 347, 354-358 (1990)). Graphite naturally has a flat surface as one of the crystal surface axes unless it is burned/scorched or modified by high voltages. The graphite electrode surface is very difficult to functionalize using traditional methods. The graphite or carbide electrodes with bound peptides of the present invention are useful, for example, for attaching catalysts, such as metallic particles, to the surface of the electrodes and can be used in biosensors and fuel cells.

Accordingly, in another embodiment a method is provided, wherein a surface-binding peptide or polypeptide is contacted with a graphite or carbide electrode having a flat surface or a smooth, curved surface, wherein the surface-binding peptide binds to the flat surface or the smooth, curved surface. Furthermore, the bound surface-binding peptide or polypeptide can be functionalized and contacted with catalyst metallic particles.

In another embodiment, a semiconductor is provided that includes a substrate composed of a semiconductor material, and a surface-binding peptide or polypeptide that is bound to a surface of the substrate, wherein the surface has a flat, or a smooth, curved geometry. The peptide or polypeptide is a surface-binding peptide or polypeptide disclosed herein. The semiconductor substrate, for example includes a Group II-VI semiconductor material. For example, the semiconductor material can include silicon, boron nitride, lead sulfide, zinc selenide, cadmium selenide, cadmium sulfide, gallium arsenide, aluminum arsenide, zinc sulfide, gallium nitrate, indium phosphate, or gallium arsenide. The semiconductor can form all or a portion of a biosensor. Surface binder peptides identified using methods disclosed herein, can be used as a connecting/binding linkers between organic materials and semiconductor (surfaces).

Semiconductor nanocrystals exhibit size and shape-dependent optical and electrical properties. These diverse properties result in their potential applications in a variety of devices such as light emitting diodes (LED), single electron transistors, photovoltaics, optical and magnetic memories, and diagnostic markers and sensors. Control of particle size, shape and phase is also critical in protective coatings such as car paint and in pigments such as house paints. The semiconductor materials can be engineered to be of certain shapes and sizes, wherein the optical and electrical properties of these semiconductor materials can best be exploited for use in numerous devices. The surface-binding peptides and polypeptides disclosed herein provide a means for binding substances to a semiconductor nanocrystal when the nanocrystal has a flat surface or a smooth, curved surface, or when the surface of the nanocrystal has a periodic, fractal, or random atomic configuration.

In another embodiment, a kit is provided that includes a surface-binding peptide or polypeptide disclosed herein. The peptide or polypeptide can bind, for example, a target atomic configuration, a flat surface, or another smooth surface, such as a smooth, curved surface. The kit, for example, can include at least two peptides or polypeptides that bind a flat surface or a smooth, curved surface. The peptides or polypeptides can be separate, or can be linked together. Furthermore, as disclosed above, the peptide or polypeptide can be associated with a nanocode. In certain aspects, the kit includes a population of nanocodes each linked to a surface-binding peptide or polypeptide disclosed herein.

In another aspect, the kit includes a phage display library and a flat, curved and/or smooth surface, or a surface with a target atomic configuration.

Although the invention has been described above, it will be understood that modifications and variations are encompassed within the spirit and scope of the invention. Accordingly, the invention is limited only by the following claims.

1. A method for identifying a peptide that binds to a surface having a target geometrical shape, comprising:
b) contacting the surface having the target geometrical shape, with a phage display library under reaction conditions, wherein the phage express a peptide; and
c) identifying peptides that bind to the surface having the target geometrical shape.

2. The method of claim 1, wherein the target geometrical shape of the surface is a flat surface.

3. The method of claim 1, wherein the target geometrical shape of the surface is a smooth, curved surface.

4. The method of claim 1, wherein the surface is hydrophobic.

5. The method of claim 1, wherein at least three rounds of biopanning are performed by repeating the contacting, identifying, and amplifying at least three times.

6. The method of claim 5, wherein during each successive round of biopanning, the reaction conditions are more stringent than the prior round.

7. The method of claim 1, further comprising amplifying the phage using a sloppy amplification reaction.

8. The method of claim 1, wherein the phage are contacted with a surface that is substantially identical in composition to the surface, but comprise a different geometrical shape.

9. The method of claim 8, wherein the substantially identical surface comprises crystals of a molecule that comprises the flat surface or the smooth, curved surface.

10. The method of claim 1, wherein the surface is a substrate for scanning probe microscopy.

11. The method of claim 1, wherein the surface comprises graphite.

12. The method of claim 11, wherein the surface comprises highly ordered pyrolytic graphite.

13. The method of claim 1, wherein the surface comprises a surfactant.

14. The method of claim 13, wherein the phage bind to both the surface and the surfactant.

15. The method of claim 1, wherein the flat surface or smooth, curved surface comprises boron nitride, lead sulfide, zinc selenide, cadmium selenide, cadmium sulfide, gallium arsenide, aluminum arsenide, zinc sulfide, gallium nitrate, indium phosphate, or gallium arsenide.

16. The method of claim 1, wherein the surface comprises mica, silicon, or annealed gold.

17. The method of claim 1, wherein the surface comprises Teflon.

18. The method of claim 1, further comprising determining the amino acid sequence of the identified peptide.

19. The method of claim 1, further comprising determining the nucleotide sequence of a phage polynucleotide that encodes the identified peptide.

20. An isolated peptide or polypeptide comprising at least two peptide units, wherein each peptide unit specifically binds a target geometrical shape, and wherein the isolated peptide or polypeptide is a recombinant peptide or polypeptide.

21. An isolated peptide or polypeptide according to claim 20, wherein the at least two peptide units are linked by a linkage other than a peptide bond.

22. An isolated peptide or polypeptide according to claim 20, wherein the peptide further comprises a nanocode.

23. An isolated peptide or polypeptide according to claim 20, wherein at least two of the at least two peptide units comprise a different amino acid sequence.

24. An isolated peptide or polypeptide according to claim 20, wherein at least one of the at least two peptide units is identified according to the method of claim 1.

25. A scanning probe microscopy (SPM) substrate, comprising a substrate comprising an SPM material, wherein the substrate has a flat surface comprising an isolated peptide or polypeptide bound thereto.

26. The SPM substrate of claim 25, wherein the SPM material comprises graphite.

27. The SPM substrate of claim 25, wherein the isolated peptide or polypeptide is associated with a biomolecule.

28. The SPM substrate of claim 25, wherein the peptide is identified according to the method of claim 1.

29. A graphite or carbide electrode having a flat surface or a smooth, curved surface, wherein a peptide is specifically bound to the surface.

30. The graphite or carbide electrode of claim 29, wherein the peptide consists essentially of between about 7 and 20 amino acids.

31. The graphite or carbide electrode of claim 29, wherein the peptide is isolated according to the method of claim 1.
